(19) United States
(12) Patent Application Publication
(10) Pub. No.: US 2026/0207658 A1
(43) Pub. Date: Jul. 23, 2026

(54) OXYGEN ENEMA COMPOSITION, ENEMA DEVICE, AND METHOD FOR PRODUCING OXYGEN ENEMA COMPOSITION

(71) Applicant: MediScience Espoir Inc., Kanagawa (JP)

(72) Inventors: Takaaki MATSUMOTO, Kawasaki-shi Kanagawa (JP); Toshikatsu HAGIWARA, Kawasaki-shi Kanagawa (JP); Miyako MATSUMOTO, Kawasaki-shi Kanagawa (JP); Seitaro SHIMIZU, Kawasaki-shi Kanagawa (JP)

(73) Assignee: MediScience Espoir Inc., Kanagawa (JP)

(21) Appl. No.: 19/567,797

(22) Filed: Mar. 16, 2026

Related U.S. Application Data
(62) Division of application No. 18/292,185, filed on Jun. 22, 2022.

(30) Foreign Application Priority Data
Jul. 28, 2021 (JP) 2021-123384

(51) Int. Cl. A61K 33/00; A61K 9/02; A61K 9/08; A61K 47/52

(52) U.S. Cl. CPC A61K 33/00; A61K 9/02; A61K 9/08; A61K 47/52

(57) ABSTRACT

A method of treatment including a step of supplying an oxygen enema composition containing an oxygen dissolved solution to an intestine from an anus of a mammal, wherein the oxygen dissolved solution has an amount of dissolved oxygen of 50 ppm or more, wherein the amount of dissolved oxygen is determined by a difference (P1−P2) between a numerical value P1 of an amount of dissolved oxygen (ppm) measured by a dissolved oxygen measurement, the method comprising the steps of: (1) adding a deoxidizing amino compound to an oxygen dissolved solution to obtain a mixture, and heating the mixture; (2) measuring a concentration of the deoxidizing amino compound contained after the step (1); and (3) calculating an amount of dissolved oxygen contained in the oxygen dissolved solution, and a numerical value P2 of an amount of dissolved oxygen (ppm) of an oxygen dissolved solution measured by a dissolved oxygen measurement method.

### CROSS-REFERENCE TO RELATED APPLICATIONS

This application is a divisional application of U.S. application Ser. No. 18/292,185 filed Jan. 25, 2024, which is a national stage application entered under 35 U.S.C. § 371 of international application no. PCT/JP2022/024834 filed Jun. 22, 2022, which claims priority to Japan Patent Application No. 2021-123384 filed Jul. 28, 2021, each of which is incorporated by reference herein in its entirety.

### TECHNICAL FIELD

The present invention relates to an oxygen enema composition, an enema device, and a method for producing an oxygen enema composition.

### BACKGROUND ART

Up to now, the present inventors have established a new method for producing an oxygen dissolved solution while conducting research on oxygen dissolved solutions. The present inventors have successfully developed an oxygen dissolved solution that maintains the dissolved oxygen in the solution stably even when heated to 100° C., and have obtained patents in the United States, China, and Japan. (Patent Literatures 1-3).

In addition, the present inventors have also established a new method for measuring the amount of dissolved oxygen ((Patent Literatures 4-6). It has also been confirmed that it contains dissolved oxygen (oxygen clathrate hydrate), which cannot be measured using conventional methods for measuring the amount of dissolved oxygen, such as the diaphragm electrode process, a fluorescence method, and a Winkler method.

By drinking this oxygen dissolved solution, dissolved oxygen is absorbed into the body. This has been confirmed to markedly increase arterial blood oxygen saturation (SpO2). Therefore, this oxygen dissolved solution is expected to be effective in treating various diseases such as chronic obstructive pulmonary disease (COPD) and depression, as well as promoting health.

Meanwhile, the new coronavirus (COVID-19) is spreading worldwide. Patients with severe cases of novel coronavirus (COVID-19) infection must be treated with ventilators or extracorporeal membrane oxygenation (ECMO) due to significant decrease in the blood oxygen levels.

### CITATION LIST

### Patent Literature

Patent Literature 1: U.S. Pat. No. 10,913,037
Patent Literature 2: CN. 107207296
Patent Literature 3: JP7087235
Patent Literature 4: JP6527161
Patent Literature 5: U.S. Pat. No. 10,018,605
Patent Literature 6: CN. 107076715

### SUMMARY OF INVENTION

### Technical Problem

Under these circumstances, it is desired to supply oxygen into the patient's body by a new method other than artificial respirators or extracorporeal membrane oxygenation (ECMO). If this new method can increase blood oxygen concentration, it is thought that it could become a new treatment method for patients with decreased respiratory function.

For example, oxygen is also absorbed through the intestines. For this reason, it is conceivable to apply oxygen-containing compositions that can be absorbed directly into the intestines, rather than by oral administration. However, at present, no method has been established that can efficiently and safely supply oxygen to a patient's intestines to the extent that it can actually be applied clinically.

The present invention has been made in view of the above circumstances, and its object is to provide an enema composition (oxygen enema composition) that can efficiently and safely absorb oxygen from the intestines. For example, the enema composition (oxygen enema composition) can be used for patients with decreased respiratory function, infants and elderly people for whom oral administration of oxygen is difficult. Another object of the present invention is to provide a treatment method using this enema composition, and a method for producing an oxygen enema composition.

### Solution to Problem

In order to solve the above problems, the present invention has the following features.

An oxygen enema composition containing an oxygen dissolved solution, the oxygen dissolved solution has the amount of the dissolved oxygen 50 ppm or more, and wherein the amount of dissolved oxygen is determined by difference (P1−P2) between a numerical value P1 of an amount of dissolved oxygen (ppm) measured by a dissolved oxygen measurement method comprising the steps of:
(1) adding a deoxidizing amino compound to an oxygen dissolved solution, and heating the resultant mixture at a temperature of 80° C. or more;
(2) measuring a concentration of the deoxidizing amino compound contained in the oxygen dissolved solution after the step (1); and
(3) calculating an amount of dissolved oxygen contained in the oxygen dissolved solution before passing through the step (1) from the concentration of the deoxidizing amino compound measured in the step (2), and
a numerical value P2 of an amount of dissolved oxygen (ppm) of an oxygen dissolved solution measured by a dissolved oxygen measurement method of any one of a diaphragm electrode process, a fluorescence method, and a Winkler method.

An enema device comprising:
an enema container and the above-mentioned oxygen enema composition.

A method for producing an oxygen enema composition comprising the step of blending an oxygen dissolved solution,
wherein the oxygen dissolved solution has the amount of the dissolved oxygen 50 ppm or more, and wherein the amount of dissolved oxygen is determined by difference (P1−P2) between a numerical value P1 of an amount of dissolved oxygen (ppm) measured by a dissolved oxygen measurement method comprising the steps of:
(1) adding a deoxidizing amino compound to an oxygen dissolved solution, and heating the resultant mixture at a temperature of 80° C. or more;
(2) measuring a concentration of the deoxidizing amino compound contained in the oxygen dissolved solution after the step (1); and
(3) calculating an amount of dissolved oxygen contained in the oxygen dissolved solution before passing through the step (1) from the concentration of the deoxidizing amino compound measured in the step (2), and
a numerical value P2 of an amount of dissolved oxygen (ppm) of an oxygen dissolved solution measured by a dissolved oxygen measurement method of any one of a diaphragm electrode process, a fluorescence method, and a Winkler method.

The treatment method of the present invention is characterized by including the step of supplying the above oxygen enema composition from the anus to the intestine (rectum or large intestine) of a mammal.

### Advantageous Effects of Invention

According to the oxygen enema composition and enema device of the present invention, oxygen can be directly supplied (enema administration) from the anus to the intestine (rectum or large intestine) of mammals including humans. Oxygen can be efficiently and safely absorbed from the intestines.

The treatment method of the present invention also includes directly supplying an oxygen enema composition to the intestines (rectum or large intestine) from the anus to mammals including humans.

According to the oxygen enema composition, enema device, and treatment method of the present invention, oxygen is efficiently and safely absorbed from the intestines. Therefore, it is effective in treating patients whose respiratory function has decreased due to, for example, the novel coronavirus (COVID-19) infection, infants, and the elderly for whom oral administration of oxygen is difficult. Furthermore, for example, by absorbing oxygen from the human intestine, the following can be achieved, for example.
Improvement of the intestinal environment that causes constipation and diarrhea.
Improvement of blood circulation.
Improvement in physical conditions such as palpitations, shortness of breath, and sensitivity to cold.
Improving beauty and health.
Improvement of diabetes, COPD, depression, etc.

### DESCRIPTION OF EMBODIMENTS

As mentioned above, the present inventors have obtained an oxygen dissolved solution in which the oxygen clathrate hydrate remains dissolved in the solution even when heated to 100° C. by improving the conventional production equipment. This oxygen dissolved solution has a dissolved oxygen amount of 50 ppm or more (for example, about 50 ppm to 200 ppm) as measured by a predetermined measuring method (the measuring method described in Patent Literatures 4-6).

The present inventors assumed that by using this oxygen dissolved solution as a component of an enema composition, oxygen can be efficiently and safely supplied from the intestines to patients with decreased respiratory function. The present inventors obtained such a new idea and completed the present invention. In addition, it has been confirmed that the oxygen dissolved solution of Patent Literatures 1 to 3 increases arterial blood oxygen saturation (SpO2) by oral administration. However, it was unclear whether or not oxygen would be sufficiently absorbed from the intestines when the oxygen dissolved solutions of Patent Literatures 1 to 3 are directly supplied from the anus to the intestines (rectum or large intestine) as an enema composition.

Hereinafter, one embodiment of the enema composition of the present invention will be described.

The present invention is an enema composition containing an oxygen dissolved solution (hereinafter sometimes referred to as “oxygen enema composition”).

The oxygen dissolved solution has the characteristics described in Patent Literatures 1-3. An oxygen dissolved solution has the amount of the dissolved oxygen 50 ppm or more, and wherein the amount of dissolved oxygen is determined by difference (P1−P2) between a numerical value P1 of an amount of dissolved oxygen (ppm) measured by a dissolved oxygen measurement method comprising the steps of:
(1) adding a deoxidizing amino compound to an oxygen dissolved solution, and heating the resultant mixture at a temperature of 80° C. or more;
(2) measuring a concentration of the deoxidizing amino compound contained in the oxygen dissolved solution after the step (1); and
(3) calculating an amount of dissolved oxygen contained in the oxygen dissolved solution before passing through the step (1) from the concentration of the deoxidizing amino compound measured in the step (2), and
a numerical value P2 of an amount of dissolved oxygen (ppm) of an oxygen dissolved solution measured by a dissolved oxygen measurement method of any one of a diaphragm electrode process, a fluorescence method, and a Winkler method.

The upper limit of the amount of dissolved oxygen determined by (P1−P2) is not particularly limited, but is, for example, 1000 ppm or less, 500 ppm or less, or 200 ppm or less.

The above measurement method utilizes the measurement method described in Patent Literatures 4-6.

For example, the deoxidizing amino compound in step (1) can be exemplified by one or more of carbohydrazide, diethylhydroxylamine, hydroxydiaminobenzene, and isopropylhydroxylamine, and among them, carbohydrazide is particularly preferred.

Furthermore, in step (1), the heating temperature of the liquid sample containing the deoxidizing amino compound is preferably in the range of 80° C. to 120° C. As a rough guide, the heating time can be approximately 3 to 4 hours when heating the oxygen dissolved solution to 80° C., and approximately 30 minutes to 2 hours when heating to 100° C.

The method for measuring the concentration of the deoxidizing amino compound in step (2) is not particularly limited, and any known method such as iodometric titration (oxidation-reduction titration) can be used as appropriate.

In step (3), the concentration of the deoxidizing amino compound measured in step (2) and the concentration of the deoxidizing amino compound in step (1) are compared, and the reaction amount of the deoxidizing amino compound is determined. Based on this, the amount of dissolved oxygen (P1) contained in the oxygen dissolved solution before passing through step (1) can be calculated.

At the time of this application, this oxygen dissolved solution is commercially available under the trade name “WOX” (registered trademark) manufactured by MediScience Espoir Inc. For its production, the production methods described in Patent Literatures 1 to 3 can be considered.

It is considered that oxygen clathrate hydrate formed by oxygen molecules and water molecules is dissolved in this oxygen dissolved solution. The term “oxygen clathrate hydrate” as used herein is a compound in which oxygen molecules are surrounded by a lattice of water molecules. Furthermore, the oxygen clathrate hydrate contained in the oxygen dissolved solution in the present invention cannot be measured by conventional methods for measuring the amount of dissolved oxygen, such as the diaphragm electrode process, the Winkler method, or the fluorescence method. It can be measured by the measurement method described in Patent Literatures 4-6.

The oxygen enema composition of the present invention is mainly composed of an oxygen dissolved solution having the above-mentioned characteristics, and preferably contains 90 to 100% by mass of the oxygen dissolved solution, more preferably, the content is 95 to 100% by mass.

Further, the oxygen enema composition of the present invention can contain known components such as glycerin as components other than the oxygen dissolved solution. Furthermore, the oxygen enema composition of the present invention includes, as other ingredients, excipients, plant extracts (for example, freeze-dried, dried, etc.), vegetable oils, diluents, solidifying agents, wetting agents, preservatives, adhesives, It may contain one or more polysaccharide agents and the like.

Further, the oxygen enema composition of the present invention preferably contains a silver complex. The silver complex is preferably either a silver complex in which silver particles and an amino acid are bonded (amino acid silver complex) or a silver complex in which silver particles and a hydroxy acid are bonded (hydroxy acid silver complex). By containing the amino acid silver complex or the hydroxy acid silver complex, the oxygen enema composition of the present invention contributes to improving the oxygen absorption effect from the intestines by the oxygen dissolved solution. Further, the oxygen enema composition of the present invention has an improved antiseptic effect by containing an amino acid silver complex or a hydroxy acid silver complex, and can be used stably for a long period of time.

The silver particles preferably have a diameter of 1 to 100 nm, more preferably 1 to 10 nm. When the silver particles have this size, a good silver complex is formed and silver ions can be effectively eluted.

The raw material for the silver particles is not particularly limited, and for example, a commercially available silver colloid dispersion can be used as appropriate. Specifically, as a commercially available silver colloid dispersion, Nano Silver Dispersion (manufactured by Nippon Aeon Co., Ltd.) can be preferably used. This nanosilver dispersion liquid is colorless and transparent and contains silver particles with a diameter of about 100 nm, and the concentration of silver particles is 10,000 ppm to 30,000 ppm.

When the oxygen enema composition of the present invention contains an amino acid silver complex, the amino acid is at least one of histidine, methionine, and cysteine. When the amino acid is histidine, methionine, or cysteine, a good amino acid silver complex is formed and silver ions can be effectively eluted. Among these, the amino acid is preferably histidine.

When the oxygen enema composition of the present invention contains a hydroxy acid silver complex, the hydroxy acid is not particularly limited, but is preferably at least one of malic acid and citric acid.

In the oxygen enema composition of the present invention, the concentration of the silver complex (amino acid silver complex or hydroxy acid silver complex) can be adjusted as appropriate within the range of 1 ppm to 50 ppm, preferably 1 ppm to 10 ppm. Note that the concentration of the silver complex (amino acid silver complex or hydroxy acid silver complex) can be measured by a known method.

Further, the enema device of the present invention includes an enema container and the oxygen enema composition of the present invention described above. The oxygen enema composition may be held within the enema container or may be held in a separate container. The enema container only needs to be able to hold the oxygen enema composition, and can employ known materials, shapes, and structures. Further, the enema device of the present invention can include an insertion portion that is inserted into the anus. This insertion section can include a tube and a nozzle disposed at the tip of the tube.

The oxygen enema composition of the present invention comprises an oxygen dissolved solution having the characteristics described above. The oxygen enema composition can be administered to humans by enema (enema administration), and can efficiently and safely absorb oxygen from the intestines. Oxygen enema compositions are typically administered to patients with decreased respiratory function due to novel coronavirus (COVID-19) infection, infants, elderly, etc. who have difficulty administering oxygen by oral administration.

That is, the treatment method of the present invention includes the step of supplying the above oxygen enema composition to the intestine from the anus of a mammal.

The mammal is, for example, a human or non-human mammal (eg, dog, cat, cow, horse, mouse, rat, monkey, rabbit, pig, etc.), but is preferably a human.

In the treatment method of the present invention, when the subject is a human, the amount of oxygen enema composition supplied to the intestines is not particularly limited, but may be, for example, in the range of 10 to 200 ml or 25 to 100 ml per administration.

Since the oxygen enema composition of the present invention mainly contains an oxygen dissolved solution (water and oxygen), the oxygen enema composition and treatment method have no adverse effects on the human body and are highly safe.

Furthermore, the method for producing an oxygen enema composition of the present invention includes the step of blending the above-mentioned oxygen dissolved solution.

Furthermore, for example, by absorbing oxygen from the human intestine, the following can be achieved, for example.
Improvement of the intestinal environment that causes constipation and diarrhea.
Improvement of blood circulation.
Improvement in physical conditions such as palpitations, shortness of breath, and sensitivity to cold.
Improving beauty and health.
Improvement of diabetes, COPD, depression, etc.

The oxygen enema composition, enema device, treatment method, and method for producing an oxygen enema composition of the present invention are not limited to the above embodiments.

### Example

Hereinafter, the oxygen enema composition, enema device, and treatment method of the present invention will be explained in detail along with Examples, but the present invention is not limited to the following Examples.

### <1> Oxygen Dissolved Solution

As the oxygen dissolved solution (oxygen water), the product name “WOX” (registered trademark) manufactured by MediScience Espoir Inc. was used.

This oxygen dissolved solution contains oxygen clathrate hydrate in water. This oxygen dissolved solution has the amount of the dissolved oxygen is 50 ppm or more (50 ppm ~1000 ppm), and wherein the amount of dissolved oxygen is determined by difference (P1−P2) between a numerical value P1 of an amount of dissolved oxygen (ppm) measured by a dissolved oxygen measurement method comprising the steps of:
(1) adding a deoxidizing amino compound to an oxygen dissolved solution, and heating the resultant mixture at a temperature of 80° C. or more;
(2) measuring a concentration of the deoxidizing amino compound contained in the oxygen dissolved solution after the step (1); and
(3) calculating an amount of dissolved oxygen contained in the oxygen dissolved solution before passing through the step (1) from the concentration of the deoxidizing amino compound measured in the step (2), and
a numerical value P2 of an amount of dissolved oxygen (ppm) of an oxygen dissolved solution measured by a dissolved oxygen measurement method of any one of a diaphragm electrode process, a fluorescence method, and a Winkler method.

### <2> Preparation of Oxygen Enema Composition

An oxygen enema composition was prepared by blending the above oxygen dissolved solution, glycerin, and an amino acid silver complex. The content of the oxygen dissolved solution in the oxygen enema composition was approximately 98 to 99% by mass.

Specifically, a commercially available silver colloid dispersion (manufactured by Nippon Aeon Co., Ltd., product name “Nano Silver Dispersion”) was diluted with an oxygen dissolved solution, and the amount of silver was adjusted to 3 ppm. This silver colloid dispersion has a silver concentration of 10,000 ppm and a diameter of silver particles of about 100 nm. Thereafter, histidine was added as an amino acid and stirred to prepare an oxygen enema composition containing an amino acid silver complex. Furthermore, glycerin (1% by mass) was blended into this oxygen enema composition.

25 to 45 ml of this oxygen enema composition was held in a syringe (enema container) of an enema device. A silicone tube is connected to the tip of the syringe, and a nozzle is connected to the tip of the silicone tube. This enema device was used to deliver the oxygen enema composition to the rectum.

Additionally, 100 ml of the oxygen enema composition was supplied from the anus to the large intestine using an intestinal cleansing device.

### <3> Blood Oxygen Concentration Measurement Test

### Test Method

The oxygen enema composition was injected into the rectum or large intestine through the anus of the subject. Fluctuations in blood oxygen concentration (SpO2) were confirmed using a commercially available pulse oximeter for 2 to 5 minutes after injection.

### Result

Table 1 shows the results of supplying the oxygen enema composition to the rectum using a syringe. Furthermore, Table 2 shows the results of supplying the oxygen enema composition from the anus to the large intestine using an intestinal cleansing device.

TABLE 1

| | Injection | | | After |
|---|---|---|---|---|
| | amount | SpO₂ before and after enema | | injection |
| Subject | ml | Before | After | Second |
| \textcircled{1} | 25 | 96 | 98 | 25 |
| \textcircled{2} | 25 | 96 | 99 | 20 |
| \textcircled{3} | 25 | 96 | 98 | 30 |
| \textcircled{4} | 25 | 96 | 98 | 25 |
| \textcircled{5} | 25 | 96 | 98 | 30 |
| \textcircled{6} | 25 | 96 | 99 | 30 |
| \textcircled{7} | 40 | 96 | 96 | 30 |
| \textcircled{8} | 40 | 96 | 97 | 120 |
| \textcircled{9} | 25 | 96 | 98 | 30 |
| \textcircled{10} | 25 | 95 | 98 | 30 |
| \textcircled{11} | 25 | 96 | 98 | 30 |
| \textcircled{12} | 25 | 96 | 97 | 30 |
| \textcircled{13} | 25 | 96 | 98 | 30 |
| \textcircled{14} | 25 | 95 | 98 | 30 |
| \textcircled{15} | 25 | 95 | 97 | 25 |
| \textcircled{16} | 25 | 96 | 97 | 25 |

TABLE 2

| | Injection | | | After |
|---|---|---|---|---|
| | amount | SpO₂ before and after enema | | injection |
| Subject | ml | Before | After | Second |
| \textcircled{1} | 100 | 96 | 96 | 180 |
| \textcircled{2} | 100 | 96 | 97 | 180 |
| \textcircled{3} | 100 | 94 | 96 | 180 |
| \textcircled{4} | 100 | 96 | 97 | 300 |
| \textcircled{5} | 100 | 95 | 98 | 300 |
| \textcircled{6} | 100 | 96 | 97 | 300 |
| \textcircled{7} | 100 | 95 | 95 | 300 |

As shown in Table 1, when the oxygen enema composition was administered from the anus to the rectum of a subject (sample), it was confirmed that the blood oxygen concentration (SpO2) increased 25 to 30 seconds after injection. It was confirmed that this increase in blood oxygen concentration (SpO2) lasted for about 10 minutes.

As shown in Table 2, when the oxygen enema composition was administered from the anus to the large intestine of the subject (sample), it was confirmed that the blood oxygen concentration (SpO2) increased 180 to 300 seconds after injection. It was confirmed that this increase in blood oxygen concentration (SpO2) lasted for about 10 minutes.

Therefore, for example, according to this oxygen enema composition, oxygen is efficiently and safely absorbed from the intestines by enema administration. Therefore, it is effective, for example, in treating patients infected with the new coronavirus (COVID-19), infants, and the elderly for whom oral administration of oxygen is difficult. Furthermore, for example, by absorbing oxygen from the human intestine, the following can be achieved, for example.
Improvement of the intestinal environment that causes constipation and diarrhea.
Improvement of blood circulation.
Improvement in physical conditions such as palpitations, shortness of breath, and sensitivity to cold.
Improving beauty and health.
Improvement of diabetes, COPD, depression, etc.

Furthermore, this oxygen enema composition is highly safe because it contains a drinkable oxygen dissolved solution as a main component.

1. A method of treatment including a step of supplying an oxygen enema composition containing an oxygen dissolved solution to an intestine from an anus of a mammal, wherein the oxygen dissolved solution has an amount of dissolved oxygen of 50 ppm or more, wherein the amount of dissolved oxygen is determined by a difference (P1−P2) between a numerical value P1 of an amount of dissolved oxygen (ppm) measured by a dissolved oxygen measurement,
the method comprising the steps of:
(1) adding a deoxidizing amino compound to an oxygen dissolved solution to obtain a mixture, and heating the mixture at a temperature of 80° C. or more;
(2) measuring a concentration of the deoxidizing amino compound contained in the heated mixture after the step (1); and
(3) calculating an amount of dissolved oxygen contained in the oxygen dissolved solution before passing through the step (1) from a concentration of the deoxidizing amino compound measured in the step (2), and
a numerical value P2 of an amount of dissolved oxygen (ppm) of an oxygen dissolved solution measured by a dissolved oxygen measurement method of any one of a diaphragm electrode process, a fluorescence method, and a Winkler method.

2. The method according to claim 1, wherein a content of the oxygen dissolved solution in the oxygen enema composition is 90 to 100% by mass.

3. The method according to claim 1, wherein the oxygen enema composition contains at least one of a silver complex in which silver particles and an amino acid are bonded, or a silver complex in which silver particles and a hydroxy acid are bonded.
